(19) United States
(12) Patent Application Publication
(10) Pub. No.: US 2013/0308755 A1
(43) Pub. Date: Nov. 21, 2013

(54) RADIATION DETECTION APPARATUS AND RADIATION DETECTION SYSTEM

(71) Applicants: Yohei Ishida, Honjo-shi (JP); Satoshi Okada, Tokyo (JP); Kazumi Nagano, Honjo-shi (JP); Keiichi Nomura, Honjo-shi (JP); Tomoaki Ichimura, Kitamoto-shi (JP); Yoshito Sasaki, Kumagaya-shi (JP)

(72) Inventors: Yohei Ishida, Honjo-shi (JP); Satoshi Okada, Tokyo (JP); Kazumi Nagano, Honjo-shi (JP); Keiichi Nomura, Honjo-shi (JP); Tomoaki Ichimura, Kitamoto-shi (JP); Yoshito Sasaki, Kumagaya-shi (JP)

(73) Assignee: CANON KABUSHIKI KAISHA, Tokyo (JP)

(21) Appl. No.: 13/983,263

(22) PCT Filed: Feb. 1, 2012
(86) PCT No.: PCT/JP2012/052798

§ 371 Date: Aug. 1, 2013

(30) Foreign Application Priority Data
Feb. 16, 2011 (JP) 2011031261

(51) Int. Cl. A61B 6/00

(52) U.S. Cl. CPC A61B 6/4208
USPC 378/62; 378/189

(57) ABSTRACT

A radiation detection apparatus comprising a sensor panel which includes a sensor array including two-dimensionally arrayed photoelectric conversion elements. A scintillator layer is arranged on the sensor panel. A scintillator protection member covers the scintillator layer upon exposing a first side surface of the scintillator layer. A housing surrounds the scintillator layer and the sensor panel. The housing includes a sealing portion to which a side surface of the sensor panel adjacent to the first side surface and the first side surface are joined by a resin.

### TECHNICAL FIELD

The present invention relates to a radiation detection apparatus and radiation detection system used in a medical diagnostic apparatus, nondestructive inspection apparatus, and the like.

### BACKGROUND ART

In some radiation imaging apparatuses, a radiation detector is contained within a frame to shorten the distance from the outer side surface of the frame to the radiation detector. Japanese Patent Laid-Open Nos. 2003-248093 and 2006-058171 disclose arrangements for shortening the distance from the detector to the side surface.

### SUMMARY OF INVENTION

The present invention provides a technique for shortening the distance from a photoelectric conversion element to the outer side surface of a housing in a radiation detection apparatus in which a sensor panel includes photoelectric conversion elements.

The first aspect of the present invention provides a radiation detection apparatus comprising a sensor panel which includes a sensor array including two-dimensionally arrayed photoelectric conversion elements, a scintillator layer which is arranged on the sensor panel, a scintillator protection member which covers the scintillator layer upon exposing a first side surface of the scintillator layer and a housing which surrounds the scintillator layer and the sensor panel, wherein the housing includes a sealing portion to which a side surface of the sensor panel adjacent to the first side surface and the first side surface are joined by a resin.

The second aspect of the present invention provides a radiation detection system comprising a radiation source which emits radiation to a subject to be inspected, radiation detection apparatus described above that detects the radiation having passed through the subject to be inspected, signal processing means for performing an image process for a signal detected by the radiation detection apparatus, and display means for displaying the signal having undergone the image process by the signal processing means.

Further features of the present invention will become apparent from the following description of exemplary embodiments (with reference to the attached drawings).

### BRIEF DESCRIPTION OF DRAWINGS

FIG. 1 is a plan view showing a radiation detection apparatus according to an embodiment of the present invention;

FIG. 2 is a sectional view showing the radiation detection apparatus according to the embodiment of the present invention;

FIG. 3 is a plan view showing a radiation detection apparatus according to another embodiment of the present invention;

FIG. 4 is a sectional view showing the radiation detection apparatus according to the other embodiment of the present invention;

FIG. 5 is a sectional view showing a radiation detection apparatus according to still another embodiment of the present invention; and

FIG. 6 is a conceptual view showing the configuration of a radiation detection system according to still another embodiment of the present invention.

### DESCRIPTION OF EMBODIMENTS

The present invention is directed to a radiation detection apparatus and radiation detection system used in a medical diagnostic apparatus, nondestructive inspection apparatus, and the like. In this specification, the radiation includes electromagnetic waves such as the α-ray, β-ray, and γ-ray in addition to the X-ray.

An embodiment of the present invention will be exemplified below with reference to the accompanying drawings.

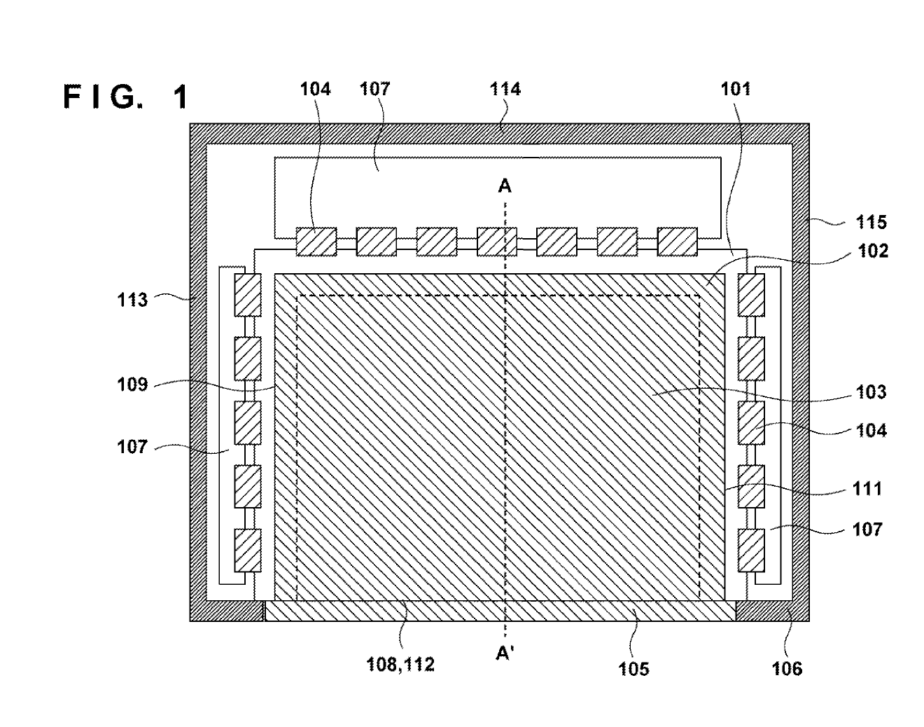

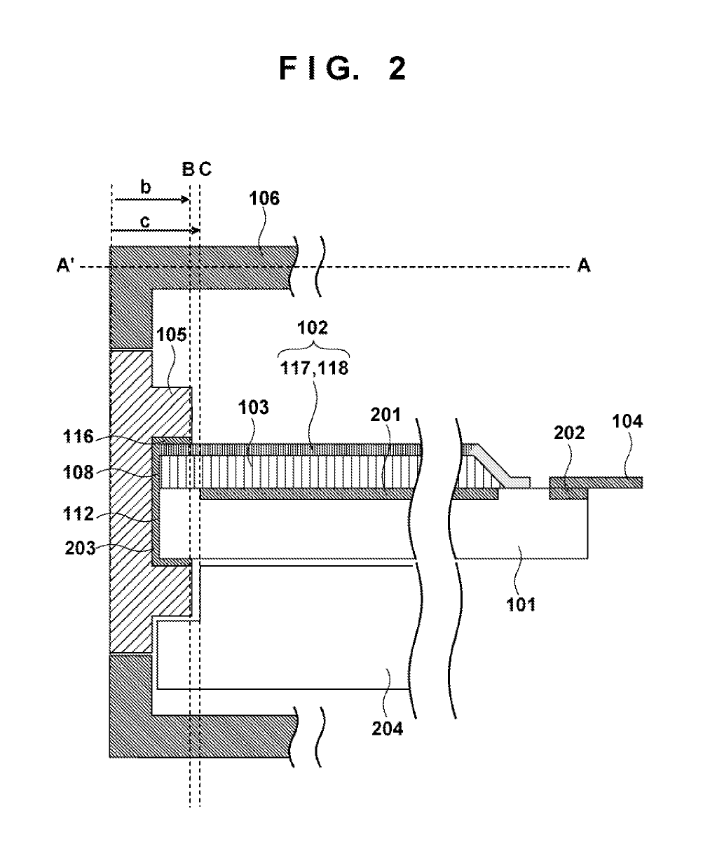

FIGS. 1 and 2 are views showing a radiation detection apparatus according to the embodiment of the present invention. A sensor panel 101 includes wiring lines (not shown), and a sensor array 201 in which photoelectric conversion elements (not shown) and TFTs (not shown) are two-dimensionally arranged on an insulating substrate made of glass, a heat-resistant plastic, or the like. The wiring lines include, for example, part of a signal line for reading out, via a TFT, a signal photoelectrically converted by a photoelectric conversion element, a wiring line for applying a bias voltage Vs to a photoelectric conversion element, or a wiring line used to drive a TFT. A signal photoelectrically converted by the photoelectric conversion element is read out by the TFT, and output to an external signal processing circuit via the signal line. The gates of TFTs arrayed in the row direction are connected to a wiring line for driving TFTs for each row, and a TFT driving circuit selects a TFT from each row.

Each external wiring line 104 of a flexible wiring board or the like is electrically connected to a connection terminal 202 via a solder, anisotropic electrically conductive adhesion film (ACF), or the like. An electrical circuit 107 is connected to the sensor panel 101 via the external wiring lines 104. The electrical circuit 107 and external wiring lines 104 form a peripheral circuit.

The photoelectric conversion element converts, into charges, light converted from a radiation by a scintillator layer 103. The photoelectric conversion element can use a material such as amorphous silicon. The structure of the photoelectric conversion element is not particularly limited, and a MIS sensor, PIN sensor, TFT sensor, or the like is appropriately usable.

A protection layer is preferably formed on the sensor panel 101 to protect the photoelectric conversion elements. Examples of the material of the protection layer are SiN, TiO₂, LiF, Al₂O₃, and MgO. Other examples are a polyphenylene sulfide resin, fluororesin, polyether ether ketone resin, liquid crystal polymer, and polyether nitrile resin. Still other examples are a polysulfone resin, polyether sulfone resin, polyarylate resin, polyamide-imide resin, polyetherimide resin, polyimide resin, epoxy resin, and silicone resin. In particular, the protection layer is desirably made of a material having high transmittance at the wavelength of light radiated by the scintillator layer 103 because light converted by the scintillator layer 103 passes through the protection layer upon radiation irradiation.

The undercoat layer of the scintillator layer may be formed to protect the sensor panel in formation of the scintillator layer. The undercoat layer of the scintillator layer uses a material resistant to a thermal process (for example, 200° C. or more for a scintillator layer having a columnar crystal structure) in the scintillator layer formation step. Examples of the material of the undercoat layer are a polyamide-imide resin, polyetherimide resin, polyimide resin, epoxy resin, and silicone resin.

A scintillator protection member 102 covers the upper surface of the scintillator layer 103. The scintillator protection member 102 is formed to efficiently collect light emitted by the scintillator layer 103 to the sensor panel 101 upon reception of radiation. Also, the scintillator protection member 102 is formed to protect the scintillator layer 103 from the external environment, and particularly humidity. The scintillator layer 103 is formed from, for example, a plurality of columnar crystals.

The scintillator layer 103 converts radiation into light that can be sensed by the photoelectric conversion elements arranged in the sensor array 201. The scintillator layer having columnar crystals can increase the resolution because light generated in the scintillator layer propagates through the columnar crystal with minimal scattering. As the material of the scintillator layer 103 which forms columnar crystals, a material mainly containing alkali halide is preferably used. For example, at least one pair of CsI and Tl, CsI and Na, CsBr and Tl, NaI and Tl, LiI and Eu, and K and Tl is used. When CsI and Tl is adopted, the scintillator layer 103 can be formed by simultaneously depositing CsI and Tl.

In the present invention, the scintillator protection member 102 and a sealing portion 105 seal the scintillator layer 103. The distance from the end of the photoelectric conversion element to the outer side surface of the sealing portion 105 included as part of a housing is preferably 5 mm or less, and more preferably 2 mm or less.

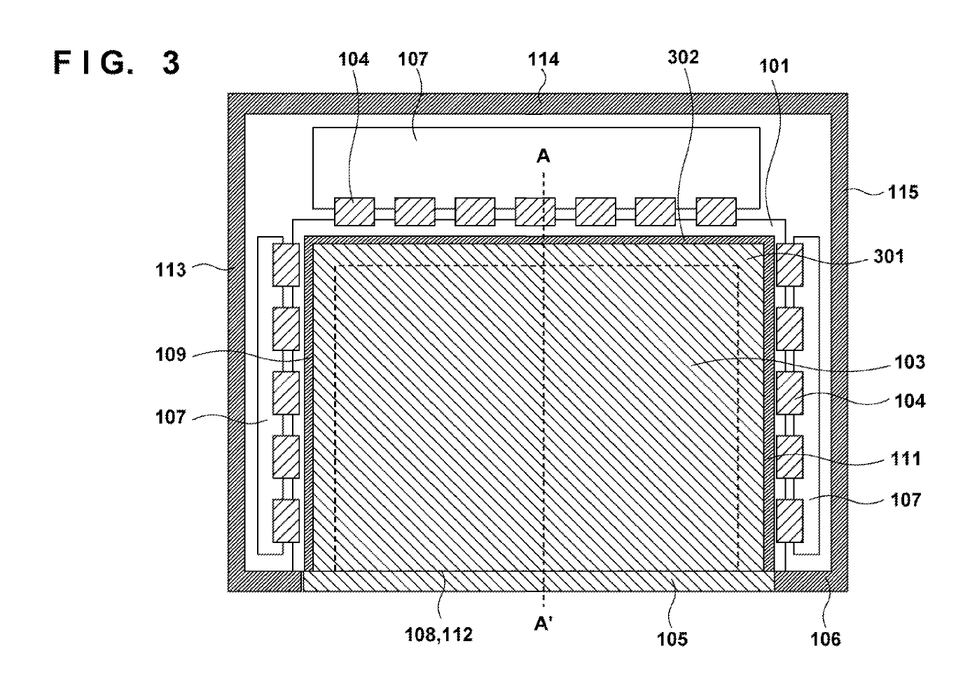

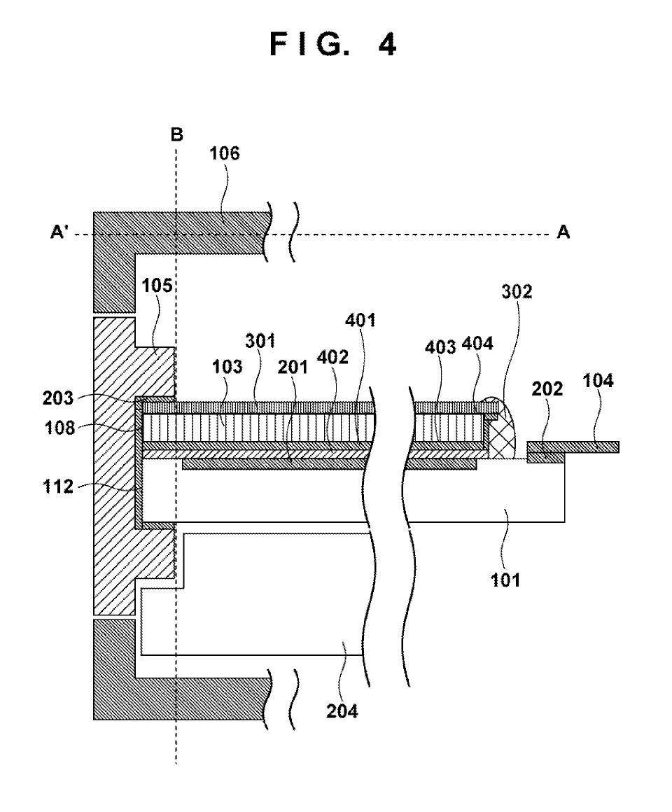

The scintillator protection member 102 has moisture resistance of preventing entrance of moisture from outside air into the scintillator layer 103, and a shock absorbing function of preventing destruction by a shock. Further, the scintillator protection member 102 has a function of increasing the light utilization efficiency by reflecting, by a scintillator reflecting layer 118, light traveling in a direction opposite to the sensor array 201 out of light converted and emitted by the scintillator layer 103 and guiding the light to the photoelectric conversion elements arranged in the sensor array 201. The scintillator protection member 102 according to the embodiment covers the scintillator layer 103 while exposing a side surface (to be referred to as the first side surface) serving as a side surface on the side of the sealing portion 105 out of side surfaces of the scintillator layer 103. In the form shown in FIGS. 1 and 2, the scintillator protection member 102 includes a scintillator protection layer 117 and the scintillator reflecting layer 118. The scintillator protection member 102 covers side surfaces of the scintillator layer 103 other than the first side surface and a surface (upper surface) facing a surface (lower surface) on the side of the sensor panel 101. To the contrary, in the form shown in FIGS. 3 and 4, the scintillator protection member is formed from a scintillator protection layer 401, and covers side surfaces of the scintillator layer 103 other than the first side surface and a surface (lower surface) facing a surface (upper surface) on the side of a base 301.

The sealing portion 105 is used for sealing of the scintillator layer 103 and as part of a housing 106, and thus is preferably made of a material having sealing property, moisture resistance, and satisfactory strength. An example of the material is a resin material such as a polyimide resin, polyether ether ketone resin (PEEK), or carbon fiber reinforced plastic (CFRP), or a metal material if degradation by the scintillator layer 103 or the external environment does not occur. Note that a portion of the sealing portion 105 to which the scintillator layer 103 is connected preferably undergoes a process of absorbing light emitted by the scintillator layer 103, for example, using a material (non-reflecting material) which absorbs emitted light or painting in black. By using such a material or process, it can be expected to suppress reflection by the side surface of the sensor panel and maintain the resolution at the end of the sensor. At the sealing portion according to the present invention, the first side surface of the scintillator layer 103 and a side surface of the sensor panel 101 that is adjacent to the first side surface of the scintillator layer 103 out of side surfaces of the sensor panel 101 are joined by a sealing resin 203. This structure can seal the first side surface of the scintillator layer 103 without using the scintillator protection member. In addition, this structure can shorten the distance from the outer side of the sealing portion 105 forming the housing 106 to the end of the sensor array 201. This structure can be preferably used as a Cassette type radiation detection apparatus which is introduced into an inspection apparatus used for mammography.

The sealing resin 203 has moisture resistance of preventing entrance of moisture into the scintillator layer 103, similar to the scintillator protection member 102 and sealing portion 105. The sealing resin 203 has a function of joining, to the sealing portion 105, the sensor panel 101, the first side surface of the scintillator layer 103, and a side surface of the sensor panel 101 that is adjacent to the first side surface of the scintillator layer 103 out of side surfaces of the sensor panel 101. The sealing resin 203 is preferably a material having high moisture resistance or a material having low water permeability. A preferable example is a resin material such as an epoxy-based resin or acrylic-based resin. A silicone-based resin, polyester-based resin, polyolefin-based resin, and polyamide-based resin are also available.

The scintillator protection layer 117 and scintillator reflecting layer 118 will be explained respectively. When a scintillator having a columnar crystal structure is used as the scintillator layer 103, the thickness of the scintillator protection layer 117 is preferably 20 to 200 μm. If the thickness is smaller than 20 μm, the scintillator protection layer 117 may not completely cover the surface roughness and splash defect of the scintillator layer 103, and the moisture resistance may deteriorate. If the thickness is larger than 200 μm, light generated by the scintillator layer 103 or light reflected by the scintillator reflecting layer 118 may scatter much more within the scintillator protection layer. As a result, the resolution and MTF (Modulation Transfer Function) of an acquired image may decrease. Examples of the material of the scintillator protection layer 117 are general organic sealing materials (for example, a silicone resin, acrylic resin, and epoxy resin), and hot-melt resins (for example, a polyester-based resin, polyolefin-based resin, and polyamide-based resin). In particular, a resin having low water permeability is desirable. As the scintillator protection layer 117, a polyparaxylylene organic film formed by CVD is preferably used. A hot-melt resin to be described below is also preferably used for the scintillator protection layer 117.

The hot-melt resin melts as the resin temperature rises, and hardens as the resin temperature drops. The hot-melt resin exhibits adhesion to other organic and inorganic materials in a heating melting state, and becomes solid and does not exhibit adhesion at room temperature. The hot-melt resin contains none of a polar solvent, solvent, and water, and does not dissolve the scintillator layer 103 (for example, a scintillator layer having an alkali halide columnar crystal structure) even if it contacts the scintillator layer 103. Thus, the hot-melt resin is used as the scintillator protection layer 117. The hot-melt resin differs from a volatile-curing adhesive prepared by a solvent application method using solvent into which thermoplastic resin has been dissolved. The hot-melt resin also differs from a chemical reaction adhesive prepared by a chemical reaction, typified by an epoxy resin. Hot-melt resin materials are classified by the type of base polymer (base material) serving as a main component, and polyolefin-, polyester-, and polyamid-based materials and the like are available. For the scintillator protection layer 117, high moisture resistance, and high light transparency of transmitting a visible ray generated by a scintillator are important.

Hot-melt resins which satisfy moisture resistance requested of the scintillator protection layer 117 are preferably a polyolefin-based resin and polyester-based resin. A polyolefin resin having low moisture absorptivity is preferably used. As a resin having high light transparency, a polyolefin-based resin is preferable. From this, a hot-melt resin containing a polyolefin-based resin is more preferable for the scintillator protection layer 117. Examples of the polyolefin resin are an ethylene-vinyl acetate copolymer, ethylene-acrylic acid copolymer, ethylene-acrylic acid ester copolymer, and ethylene-methacrylic acid copolymer. Other examples are an ethylene-methacrylic acid ester copolymer and ionomer resin. At least one material selected from these polyolefin resins is preferably selected and contained as a main component.

A hot-melt resin mainly containing an ethylene-vinyl acetate copolymer is Hirodine 7544 (available from Hirodine Kogyo). A hot-melt resin mainly containing an ethylene-acrylic acid ester copolymer is 0-4121 (available from Kurabo Industries). A hot-melt resin mainly containing an ethylene-methacrylic acid ester copolymer is W-4110 (available from Kurabo Industries). A hot-melt resin mainly containing an ethylene-acrylic acid ester copolymer is H-2500 (available from Kurabo Industries). A hot-melt resin mainly containing an ethylene-acrylic acid copolymer is P-2200 (available from Kurabo Industries). A hot-melt resin mainly containing an ethylene-acrylic acid ester copolymer can be Z-2 (available from Kurabo Industries).

The scintillator reflecting layer 118 has a function of increasing the light use efficiency by reflecting light traveling in a direction opposite to the sensor array 201 out of light converted and emitted by the scintillator layer 103 and guiding the light to the photoelectric conversion elements arranged in the sensor array 201. The scintillator reflecting layer 118 further has a function of preventing external light other than one generated by the scintillator layer 103 from entering the sensor array 201, and preventing noise from entering the photoelectric conversion element.

The scintillator reflecting layer 118 preferably uses a metal foil or metal thin film, and its thickness is preferably 1 to 100 μm. If the thickness is smaller than 1 μm, a pinhole defect is readily generated in formation and the light-shielding property becomes poor. If the thickness exceeds 100 μm, the radiation absorption becomes large, which may increase the dose by which an object is exposed to radiation. Further, it may become difficult to cover the step between the scintillator layer 103 and the surface of the sensor panel without a gap. The material of the scintillator reflecting layer 118 can be a metal material such as aluminum, gold, copper, or an aluminum alloy. In particular, aluminum and gold are preferable as high-reflectivity materials.

### Example 1

A radiation detection apparatus according to Example 1 of the present invention will be described.

A sensor panel 101 shown in FIGS. 1 and 2 is prepared. For example, the sensor panel 101 is formed as follows. First, an amorphous silicon semiconductor thin film is formed on an insulating substrate made of glass or the like, and wiring lines and a sensor array 201 including a plurality of photoelectric conversion elements and a plurality of TFTs are formed on the film. Then, a silicon nitride sensor protection layer and polyimide resin are applied and cured on a surface of the sensor panel on which the photoelectric conversion elements are formed, thereby forming a scintillator undercoat layer.

After the scintillator undercoat layer undergoes a process of improving adhesion, a scintillator layer 103 made of, for example, an alkali halide phosphor (for example, CsITl (thallium-activated cesium iodide)) having a columnar crystal structure is formed. At this time, the scintillator layer 103 is formed to have an average film thickness of 0.2 mm in the formation region. The scintillator layer 103 is formed by, for example, vacuum evaporation, and the formation position is restricted to cover the entire sensor array 201 and not to form the scintillator layer 103 at portions where a scintillator protection member 102 and external wiring line 104 are to be formed in subsequent steps. The microstructure of the scintillator layer 103 formed in this way is observed using a scanning electron microscope (SEM) to find that a plurality of columnar crystals are formed and a gap exists between the columnar crystals.

After that, the scintillator protection member 102 is formed to cover the scintillator layer 103. The scintillator protection member 102 is formed as follows. An Al film is formed in advance as a reflecting layer on a PET (polyethylene terephthalate) sheet. A scintillator protection layer of a hot-melt resin made of a polyolefin resin is transferred and adhered using a heating roller to the reflecting layer surface of the film sheet covered by the Al film, thereby obtaining a three-layered film sheet. The three-layered film sheet is arranged to cover the scintillator layer 103 on the sensor panel while the periphery of the three-layered film sheet overlaps the upper surface of the sensor panel 101. The resultant structure is heated and pressed by a vacuum lamination process, welding the scintillator protection member. The three-layered film sheet serving as the scintillator protection member 102 is therefore arranged and fixed on the scintillator layer 103. At this time, the scintillator layer 103 is covered with the three-layered film sheet serving as the scintillator protection member 102, and the sensor panel 101. Further, a peripheral portion of the scintillator protection member 102 where the scintillator layer 103 is not formed is compression-bonded by a bar type thermo-compression bonding head, improving the sealing performance of the scintillator protection member 102. For example, the thermo-compression bonding process is performed at a pressure of 1 to 10 kg/cm² and a temperature higher by 10 to 50° C. than the melting start temperature of the hot-melt resin for 1 to 60 sec.

A side of the sensor panel 101 on which the scintillator layer 103 and scintillator protection member 102 are formed, which is to be joined to a sealing portion 105, is cut using a cutting tool such as a diamond saw. The section of the scintillator layer 103 that is exposed after cutting is defined as a first side surface 108. The scintillator protection member 102 protects remaining side surfaces 109, 110, and 111 of the scintillator layer 103. A sealing resin 203 join the sealing portion 105 with the first side surface 108 of the scintillator layer and a second side surface 112, which is adjacent to and runs parallel to the first side surface 108 that is one of the side surfaces of the sensor panel 101, thereby protecting the exposed portion of the scintillator layer 103. When viewed from above the sensor panel (from top to bottom in FIG. 2), the sealing portion 105 and sealing resin 203 seal a portion up to a dotted line B in FIG. 2, as shown in FIG. 2. A dotted line C indicates the end of the photoelectric conversion element. The cutting position of the sensor panel 101 and the shape of the sealing portion 105 may be designed to set the sealed portion at a position where the sealed portion does not overlap the sensor array 201 formed on the sensor panel 101. That is, a distance b from the outer side surface of the housing in FIG. 2 to the end B of the sealing portion 105 and sealing resin 203, and a distance c from the outer side surface of the housing to the end C of the sensor array 201 may be designed to have a relation of b<c.

Thereafter, electrical components are mounted on the sensor panel 101 connected to the sealing portion 105. The external wiring lines 104 and mounting components are connected to connection terminals 202 serving as the signal input/output portions of the sensor panel. Finally, a housing 106 is arranged to protect the sensor panel 101. The housing 106 may be formed to surround the peripheries of the scintillator layer and sensor panel, partially have an opening to place the sealing portion 105, and form a space between the upper surface of the scintillator and the housing. By placing the sealing portion 105 in the opening, the sealing portion 105 can be used as part of the housing 106. The sealing portion 105 and housing 106 are joined with an adhesive. By these steps, a radiation detection apparatus according to the present invention is manufactured.

In Example 1, the distance from the outer side surface of the housing 106 having the sealing portion 105 as its part to the end of the sensor array 201 on the sensor panel 101 was 1.5 mm. This radiation detection apparatus is usable as a cassette type radiation detection apparatus which is introduced into an inspection apparatus used for mammography.

### Example 2

Example 2 will exemplify a radiation detection apparatus which is manufactured by the same method as that in Example 1 after a scintillator layer and scintillator protection member are formed up to the panel end without cutting the sensor panel, which is executed in Example 1.

When depositing a scintillator layer on a sensor panel 101 on which a protection layer is formed, the scintillator layer on a side A′ in FIG. 2 is formed to the end of a side surface of the sensor panel on the side A′. The scintillator layer is deposited after a mask is set at a portion where deposition is unnecessary. Example 2 suffices to execute deposition without setting the mask on a surface on the side of the side surface of the sensor panel serving as the sealing portion side. Then, a scintillator protection member 102 is formed. At this time, the scintillator protection member 102 is formed so that a first side surface 108 of the scintillator layer is finally exposed without covering the scintillator layer on the side of the side surface with the scintillator protection member.

After that, a sealing portion 105 is joined by the same method as that of Example 1, sealing the first side surface 108 on which a scintillator layer 103 is exposed. At this point, in order to protect from degradation due to humidity, processes from the forming process of the scintillator layer 103 to the joining process of the sealing portion 105 are preferably performed in a low-humidity environment, and the time required from the forming process of the scintillator layer 103 to the joining process of the sealing portion 105 should be kept within 180 min.

Similar to the apparatus in Example 1, the radiation detection apparatus manufactured in Example 2 could shorten the distance from an outer side surface of the housing on the side of the sealing portion 105 to the end of a sensor array 201 to be 1.5 mm. Since degradation of the end of the scintillator layer is suppressed, a high-resolution inspection image can be acquired.

### Example 3

Example 3 will exemplify a radiation detection apparatus which is manufactured by a method of forming a scintillator layer 103 on a scintillator formation base 301 for supporting one surface of the scintillator layer shown in FIGS. 3 and 4, and then mounting the scintillator layer 103 and base 301 on a sensor panel 101.

First, a sensor panel 101 on which a scintillator protection member is formed is prepared by the same method as that of Example 1. Then, the base 301 for forming a scintillator is prepared, as shown in FIGS. 3 and 4. An aluminum plate is used as the base 301, and the base 301 also serves as a scintillator reflecting layer 118 in FIG. 2. A protection layer (not shown) is formed on the base 301. The base 301 functions as the scintillator protection layer.

A scintillator layer 103 is formed on one surface of the base 301 by the same method as that of Example 1, manufacturing a scintillator panel. At this time, similar to Example 1, a mask is used to form the scintillator layer 103 only in a necessary region. After that, a scintillator protection layer 401 is formed on a surface of the scintillator layer 103 that is opposite to a surface on the side of the base 301, and the side surface of the scintillator layer 103. A 20 μm thick hot-melt resin is available for the scintillator protection layer 401.

After forming the scintillator panel, the surface 403 of the scintillator layer 103, which is opposite the surface 404 of the scintillator layer 103 supported by the formation base 301, is adhered to the sensor panel 101. For adhesion, an adhesive layer 402 such as an acrylic adhesive may be used. The thickness of the adhesive layer 402 is preferably 25 μm. After adhesion, a degassing process is performed and bubbles existing between the scintillator panel and the sensor panel are removed.

Next, a first side surface 108 side, which is a side to be joined to a sealing portion 105 of the scintillator panel, and a second side surface 112 side, which is adjacent to and runs parallel to said first side surface 108 that is one of the side faces of the sensor panel 101 attached to the scintillator panel by an adhesive layer 402, are cut. Cutting is performed, for example, using a cutting tool such as a diamond saw. The side surface after cutting is sealed by the sealing portion 105 and a sealing resin 203.

Thereafter, a radiation detection apparatus is manufactured by the same method as that of Example 1.

### Example 4

Example 4 will exemplify a radiation detection apparatus which is manufactured by forming a scintillator on a scintillator formation base by the same method as that of Example 3, and then joining a sealing portion without execution of the cutting step. First, a scintillator layer 103 is formed on a scintillator formation base 301 by the same method as that of Example 3. In this case, scintillator layer is formed to an end of the base 301 serving as a side surface to later be joined to a sealing portion 105, and formation of the scintillator layer is restricted such that components can be mounted on the remaining three sides, later at mounting time.

Then, a scintillator protection layer 401 is formed on the scintillator layer 103. Similar to Example 3, a hot-melt resin is used for the scintillator protection layer 401. At this time, the scintillator protection layer 401 cover the scintillator layer 103 upon exposing a first side surface 108 of the scintillator layer 103. Thereafter, a radiation detection apparatus as shown in FIG. 4 is manufactured through the same steps as those of Example 3.

### Example 5

Example 5 will exemplify a structure in which a sealing portion 105, scintillator layer 103, and sensor panel 101 are joined more strongly.

As shown in FIG. 2, a first side surface 108 on which the scintillator layer 103 is exposed and a second side surface 112, which is adjacent to and runs parallel to said first side surface 108 that is one of the side faces of the sensor panel 101, are joined to the sealing portion 105 by sealing resin 203. Further, outer surfaces 116 adjacent to the first side surface 108 of the scintillator and the second side surface 112 of the sensor panel 101 are joined to the sealing portion with the resin.

### Example 6

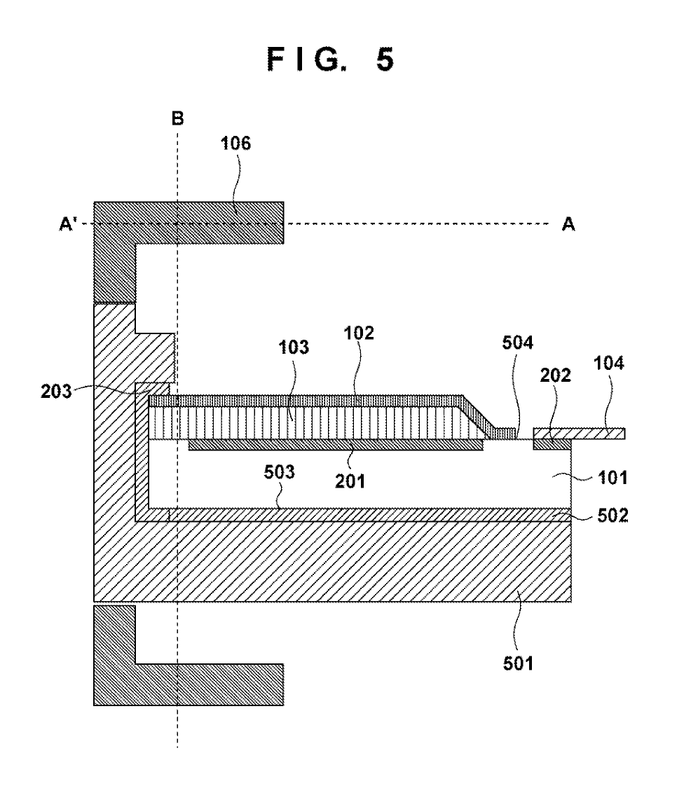

Example 6 will exemplify a structure in which a sealing portion 501 also serves as a support panel for supporting a sensor panel 101, as shown in FIG. 5. A scintillator layer 103 and scintillator protection member 102 are formed on the sensor panel 101 by the same method as that of Example 1. After this, a surface 503, different from a sensor panel surface 504, on which the scintillator layer 103 is formed, is adhered to the sealing portion 501, which also serving as the support panel of the sensor panel 101, by adhesion layer 502. The adhesion layer 502 may be made of the same material as that of a sealing resin 203 such as an epoxy resin, or another material such as a sheet-like adhesion material (adhesive sheet).

Then, a radiation detection apparatus having a shape shown in FIG. 5 is manufactured through the same steps as those of Example 1. Since the support panel formation step is omitted in the radiation detection apparatus manufactured in Example 6, the manufacturing process can be simplified.

### Example 7

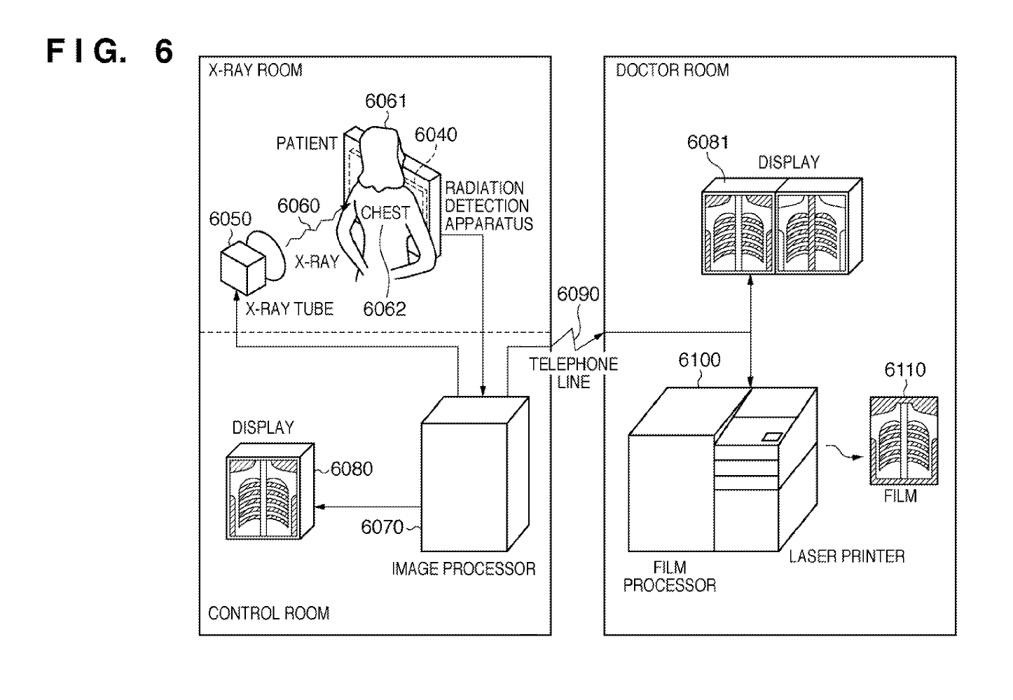

FIG. 6 exemplifies an application of the radiation detection apparatus according to the present invention to an X-ray diagnosis system. The radiation detection apparatus includes an X-ray tube 6050 as a radiation source which emits a radiation to a subject to be inspected. A generated X-ray 6060 passes through a chest 6062 of a patient or subject 6061, and enters a radiation detection apparatus (image sensor) 6040 as shown in FIG. 6. The entering X-ray contains internal information of the patient or the subject 6061. The scintillator (scintillator layer) emits light in correspondence with the entrance of the X-ray, and the photoelectric conversion elements of the sensor panel photoelectrically convert the light, obtaining electrical information. This information is digitally converted, undergoes an image process by an image processor 6070 serving as a signal processing means, and can be observed on a display 6080 serving as a display means in the control room.

This information can be transferred to a remote place by a transmission processing means such as a telephone line 6090, and can be displayed on a display 6081 serving as a display means in a doctor room or the like at another place or saved on a recording means such as an optical disk, allowing a doctor at a remote place to make a diagnosis. The information can also be recorded on a film 6110 by a film processor 6100 serving as a recoding means. In this way, the radiation detection system can be configured.

### INDUSTRIAL APPLICABILITY

As described above, the present invention is applicable to the medical field and the like, and is also effectively applied to another purpose such as nondestructive inspection.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-031261, filed Feb. 16, 2011, which is hereby incorporated by reference herein in its entirety.

1. A radiation detection apparatus comprising:
a sensor panel (101) which includes a sensor array (201) including two-dimensionally arrayed photoelectric conversion elements;
a scintillator layer (103) which is arranged on said sensor panel;
a scintillator protection member (102) which covers said scintillator layer upon exposing a first side surface (108) of said scintillator layer; and
a housing (106) which surrounds said scintillator layer and said sensor panel,
wherein said housing (106) includes a sealing portion (105) to which a second side surface (112) of said sensor panel adjacent to the first side surface (108) and the first side surface (108) are joined by a resin.

2. The apparatus according to claim 1, characterized in that said scintillator protection member covers side surfaces of the scintillator layer other than the first side surface and a first surface of the scintillator layer facing a second surface of the scintillator layer on the side of the sensor panel.

3. The apparatus according to claim 1, characterized in that said scintillator protection member includes a base for supporting first surface of the scintillator layer facing a second surface of the scintillator layer on the side of the sensor panel and a layer which covers the second surface and side surfaces of the scintillator layer other than the first side surface, and the second surface is adhered to said sensor panel.

4. The apparatus according to claim 1, characterized in that the outer surfaces of the said scintillator layer and said sensor panel, that are adjacent to the first side surface and the second side surface, are joined to said sealing portion with resin.

5. The apparatus according to claim 1, characterized in that a distance from an end of said sensor panel on a side of said sealing portion to an outer side surface of said housing on the side of said sealing portion when said sealing portion is connected to said sensor panel is not larger than 5 mm.

6. The apparatus according to claim 1, characterized in that a distance from an end of said sensor panel on a side of said sealing portion to an outer side surface of said housing on the side of said sealing portion when said sealing portion is connected to said sensor panel is not larger than 2 mm.

7. The apparatus according to claim 1, characterized in that said scintillator layer has a columnar crystal structure, and the columnar crystal structure essentially consists of at least one pair selected from the group consisting of CsI and Tl, CsI and Na, CsBr and Tl, NaI and Tl, LiI and Eu, and K and Tl.

8. A radiation detection system comprising:
a radiation source (6050) which emits radiation to a subject to be inspected;
a radiation detection apparatus (6040) according to claim 1 that detects the radiation having passed through the subject to be inspected;
signal processing means (6070) for performing an image process for a signal detected by said radiation detection apparatus; and
display means (6080, 6081) for displaying the signal having undergone the image process by said signal processing means.
